(19) United States
(12) Patent Application Publication
(10) Pub. No.: US 2003/0054424 A1
(43) Pub. Date: Mar. 20, 2003

(54) Rapid and sensitive method for measuring PCB contamination

(76) Inventors: Randy L. Allen, Apex, NC (US); Thomas N. Stewart, Durham, NC (US)

Correspondence Address: Michael N. Mercanti, ROBERTS & MERCANTI, L.L.P., P.O. Box 3156, Newark, NJ, 07103-0156, US

(21) Appl. No.: 10/107,867

(22) Filed: Mar. 27, 2002

Related U.S. Application Data
(60) Provisional application No. 60/281,869, filed on Apr. 5, 2001.

(51) Int. Cl. G01N 33/53; G01N 33/537; G01N 33/543

(52) U.S. Cl. 435/7.92; 435/7.1

(57) ABSTRACT

Immunoassay methods and standards are provided for determining the presence of polychlorinated biphenyl compounds, in a sample of interest:

(a) extracting PCB compounds from the sample into a nonpolar solvent that will dissolve the PCB, to produce an analyte extract,

(b) contacting the analyte extract with an mAB with specific reactivity towards PCB compounds under conditions suitable for the monoclonal antibody to bind to the PCBs present in the sample,

(c) measuring mAB that is selectively bound to PCB compounds and congeners thereof, to provide a binding level,

(d) relating the binding level of the mAB of step (c) with binding levels of the same mAB with a control composition, wherein the control composition comprises a plurality of PCB of compounds of known concentration, and then determining concentrations of PCB compounds in the sample.

### CROSS REFERENCE TO RELATED APPLIATIONS

This application claims the benefit of priority from U.S. Provisional Patent Application of U.S. Serial No. 60/281,869, filed on Apr. 5, 2001, the contents of which are incorporated by reference herein in its entirety.

### FIELD OF THE INVENTION

The invention relates to rapid and sensitive immunoassay methods for measuring polychlorinated biphenyl compounds, and related congeners and metabolites thereof, in biological materials, including tissues of plants and animals having a significant lipid content. The invention also relates to compositions and methods for standardizing the inventive immunoassays.

### BACKGROUND OF THE INVENTION

Polychlorinated biphenyls (“PCBs”) were formerly employed by industry to produce, among other products, plastics and insulators. Production of these compounds was ultimately banned when the toxic nature of these compounds, and their stability and persistence in the environment, made it clear that these materials are a serious environmental toxin.

However, PCBs remain present in industrial materials throughout the world. For instance, PCB contamination often results from the escape of PCBs employed within certain types of large electrical transformers, when the transformers overheat and burn, or when such devices leak after being discarded in landfills. Thus, the problem of PCB contamination of the environment remains ongoing, and there are concerns about the safety of the food supply.

Recent studies have reported the presence of PCBs in food, Alcock et al. 1998 (1) and animal feed, Bernard et al. 1999 (5). Exposure to PCBs has been linked to a wide variety of toxicological and biological effects in animals and humans. These include immunosuppression, induction of undesirable enzyme activity, tumor promotion, hepatotoxicity, and reproductive and developmental toxicity. Seegal et al. 1992 (22), Kimbrough 1993 (20), Battershill 1994 (4), Cheng and Hsu 1994 (7). In addition, PCBs tend to accumulate in the fatty tissue of living organisms due to their high lipophilicity and high degree of persistence. Erickson, 1997 (11). In fact, PCBs are among the most stable organic compounds known. Most recently, Bernard et al 1999 Id, have reported the widespread poisoning of chicks that consumed contaminated feed.

Thus, there is an urgent need for rapid, sensitive and cost effective methods for detecting PCBs in substances, including biological materials that are used as human or animal foods, food additives, medicinals, and a myriad of related materials, all of which have the potential to poison desirable animal populations or humans.

There is also an important need to for rapid, sensitive and economical methods for detecting the presence of undesirable levels of PCBs in animal and human tissues, including blood, milk and derivatives thereof, as the first step in identifying situations requiring remediation and/or ongoing monitoring, as well as for the rapid diagnosis of individuals requiring medical attention for acute or chronic PCB poisoning.

In order to appreciate the difficulties inherent in achieving this goal, it will be helpful to consider the nature of PCBs. These are a series of synthetic compounds that differ only in the position and number of chlorine atoms. Commercial PCBs were produced as mixtures of PCB congenors containing 209 different isomeric forms. These mixtures were distributed commercially under the commercial name Aroclor. A number assigned to the Aroclor designation indicates average percent chlorination of the PCB congenors in the product. Thus, Aroclor 1260 contains PCBs with an average chlorination of 60%, Aroclor 1254 has an average chlorination of 54%, Aroclor 1248 has an average chlorination of 48%, Aroclor 1242 has an average chlorination of 42%, and Aroclor 1232 has an average chlorination of 32%. The only one of the important toxic Aroclors which does not follow the above rule is Aroclor 1016, which has an average chlorination of 41%. Studies have indicated that the highly chlorinated PCBs are the most toxic.

It should also be noted that the composition of PCBs varies from individual product to individual product, from manufacturer to manufacturer within the same product, and even from lot to lot within the same product. This problem is compounded when measuring biological materials, such as foods, because studies have shown that the tissues of living organisms tend to selectively accumulate specific PCB congeners that do not resemble commercial samples. Schwartz and Stalling, 1987 (23); Jordan and Feeley 1999 (19). This is believed to result from preferential in vivo transformation of ingested PCBs, as well as a relatively more rapid clearance of the less chlorinated congeners.

Further, in such biological materials, substantially all of the PCBs are present in the fat or oil fraction. Thus, any analysis method must be accurate in the presence of fats, or there must be a rapid and effective method of extracting the PCBs from the fats, for analysis by immunoassay. For these and other reasons, the development of a rapid and accurate immunoassay to measure PCBs in biological materials such as foods, has proved elusive.

Before the present invention, the “gold standard,” for determinations of PCBs in biological materials, and especially in fatty food samples, required gas chromatography (“GC”) methods. CEN, 1997 (6). These GC-based analysis methods generally include an extraction step using organic solvents, such as hexane or methylene chloride, followed by a clean-up step by column fractionation, and a final gas chromatographic separation and analysis. A mass spectrometer (“MS”) can be employed with the GC to enhance accuracy. While highly accurate, GC and GC/MS analysis is limited by slow throughput of test samples and high cost. A GC and/or GC/MS apparatus handles only one sample at a time, requires 40-60 minutes per test sample, and highly trained technical personnel are required to perform the testing and maintain the equipment.

Immunoassays have proven to be sensitive, accurate, and cost-effective analytical tools for detecting many environmental contaminants, including PCBs, Allen et al. 1992 (2), Mapes et al. 1993 (21); Withers et al. 1995 (25). However, until the present invention, no satisfactory solution to the requirements for an immunoassay for PCBs in biological materials having a rapid throughput, accuracy and economical operation have emerged, despite great effort.

For example, the detection of PCBs in milk and blood by radioimmunoassay has been reported by W. H. Newsome et al., 1981, Intern. J. Environ, Anal. Chem. 10:295-304. Antisera was elicited in rabbits using a succinamide linking arm to the hapten (2-amino-2′,4,4′,5,5′-pentachlorobiphenyl) and the radiotracer was 2-[L¹²⁵iodo]-2′,4,4′,5,5′-pentachlorobiphenyl. The minimum sensitivity reported was about 0.1 ng for Aroclors 1260 and 1254, but lower Aroclors were not detected with the same sensitivity.

Stark, U.S. Pat. No. 4,456,691, describes the preparation of polyclonal antibodies elicited to PCBs using Aroclor 1254 that was aminated, diazotized and coupled to bovine serum albumin (BSA). The antisera was evaluated by radioimmunoassay (“RIA”). While RIAs provide acceptable sensitivity, these assays are slower and more expensive than other types of immunoassays, since RIAs require a higher degree of technical expertise, are more cumbersome that other immunoassay methods, require expensive equipment, and involve the handling and disposal of radioactive tracer materials.

Friedman, et al. 1998, 1999 (U.S. Pat. Nos. 5,834,222, and 5,858,692), incorporated by reference herein in their entireties, have described methods and antibodies for detecting PCB contamination of soils and the like. However, unlike food, soils typically do not contain substantial levels of fats or oils, and the PCB contamination of soils has not been reported to involve the degree of selective alteration of the congener mix that occurs within living organisms. While Friedman et al. teach a useful anti-PCB monoclonal antibody, these authors do not provide methods for testing PCBs in the much more challenging situation involving biological materials, particularly those containing lipids.

The methods described by Friedman, et al. for extracting PCBs from mineral oils, such as, transformer oils, non-PCB dielectrics, motor oils, silicone oils, fuel oils, organic solvent waste streams of synthetic processes, chlorinated solvents from the dry cleaning and electronics industries, gas pipeline condensates and phthalate ester based oils, as well as aqueous based paints and condensates that contain oil-based contaminants, are not generally applicable to extracting PCBs from the lipid content of biological materials. Moreover, the sample processing for the oil-based contaminants described by Friedman et al., is extremely complex and time-consuming, rending the procedure not suitable for rapid testing.

More recently, Zajicek, et al. , 2000 (Chemosphere, 40:539-548) described the application of an immunoassay to determine PCB contamination levels in fish. The procedure of Zajicek et al. utilized sample processing procedures optimized for GC, requiring large amounts of biological matrix and extensive cleanup. The primary shortcoming is reflected in the poor correlation (validation) with the GC method. The authors recognized poor correlation (r2=0.75) and suggested as a remedy the use of a different standard curve. The standard that they suggested for future work (Aroclor 1254, by itself) would also not be suitable, particularly in view of the need for multicomponent standards as confirmed by the present invention.

As a result, the testing or screening of biological materials for PCB compounds has continued to require the relatively slow and expensive gas chromatography—mass spectroscopy methods. Thus, there remains a longstanding and urgent need in the art for rapid, sensitive and economical immunoassay for detecting PCBs and derivatives and metabolites thereof in biological materials.

### SUMMARY OF THE INVENTION

Accordingly, the present invention provides methods, standards and kits for rapid, sensitive and economical detection of PCB compounds in biological materials, and particularly in biological materials that contain significant proportions of fats and oils. The inventive immunoassay employs a monoclonal antibody, or mAB, able to selectively bind to multiple PCB congeners or Aroclors, and further provides for a mixture of PCB congeners suitable for standardizing the immunoassay. The invention also provides for control of the PCB determinations by correlating the immunoassay binding to GC/MS analysis of materials similar to those being tested.

Thus, an immunoassay is provided for determining the presence of polychlorinated biphenyl compounds, in a sample of interest, the process comprising the steps of:

(a) extracting polychlorinated biphenyl compounds present in the sample of interest into a nonpolar solvent to produce an analyte extract,

(b) contacting the analyte extract with a monoclonal antibody (“mAB”), e.g, for a period of about 5 to about 60 seconds, wherein the mAB has a specific reactivity towards PCB compounds, e.g., AROCLORs 1260, 1254, 1248, 1242, 1232, 1016 and 1221 that is substantially the same as the monoclonal antibody produced by clone ATCC No. HB-12421, and the reaction is conducted under conditions suitable for the monoclonal antibody to bind to PCB compounds present in the sample,

(c) measuring mAB that is selectively bound to polychlorinated biphenyl compounds and congeners thereof, to provide a binding level,

(d) relating the binding level of the mAB of step (c) with binding levels of the same mAB with a control composition, wherein the control composition comprises a plurality of polychlorinated biphenyl compounds of known concentration, and then determining concentrations of PCB compounds in the sample of interest; provided that the nonpolar solvent solubilizes polychlorinated biphenyl compounds.

Optionally, the sample of interest comprises lipids, as is very often found in biological materials, such as food products. When the analyte of interest includes lipids, the PCBs are preferably extracted into the nonpolar solvent from the lipid phase by a process comprising:

(i) treating the sample of interest by homogenizing the sample, heating the sample, and/or combinations thereof, in the presence of the nonpolar solvent;

(ii) separating the nonpolar solvent from the treated sample of interest;

(iii) removing dissolved or suspended lipid from the nonpolar solvent to provide an analyte extract that includes the nonpolar solvent and any extracted PCBs.

Preferably, the PCB compounds are extracted into the nonpolar solvent from the lipid phase of the sample of interest by a process comprising

(i) separating the lipid phase from the sample of interest homogenizing the sample, heating the sample, and/or a combination thereof, and collecting the separated lipid,

(ii) dissolving the separated lipid in a nonpolar solvent, for example, by mixing the nonpolar solvent with a strongly acidified polar solvent, under conditions effective to render the lipid phase soluble or miscible in the polar solvent;

(iii) removing dissolved or suspended lipid from the nonpolar solvent to provide an analyte extract that includes the nonpolar solvent and any PCB compounds, e.g., by removing the above mentioned acidified polar solvent carrying the solubilized lipid. Preferably, the nonpolar solvent is substantially immiscible with the strongly acidic polar solvent.

Preferably, the strongly acidic polar solvent is aqueous concentrated sulfuric acid. Useful nonpolar solvents include, for example, solvents that are straight or branched alkyl, substituted or nonsubstituted, an aryl, substituted or nonsubstituted, and combinations thereof, wherein the alkyl moiety ranges in size from about C₃ through about C₂₀. Simply by way of example, the nonpolar solvent includes, hexane, isooctane, heptane, ethyl acetate, diisopropyl ether, diethyl ether, dichloromethane, dichloroethane, cyclopentane, cyclohexane, chloroform, carbon tetrachloride, n-butanol, butyl acetate, benzene, pentane, methyl t-butyl ether, trichloroethylene, toluene, ether and combinations thereof.

While any suitable art-known immunoassay format is readily employed, the binding level of step (c), as described supra, is determined by conducting a dissociation-enhancement immunoassay, e.g., a lanthanide fluoro-immunoassay, by:

(i) incubating the analyte extract with the anti-PCB mAB in chambers coated with capture antibody,

(ii) washing the microtiter wells, and

(iii) adding a signal producing reagent, e.g., Europium, and determining the signal.

The immunoassay according to the invention is readily applied to determining the PCB compounds when the sample of interest is a foodstuff for human or animal consumption. Such foodstuffs are, e.g., derived from a vegetable or animal source. The standardized control samples include PCB compounds of a type and approximate concentration as determined by gas chromatography and mass spectroscopy to be present in the type of foodstuff to be tested.

The immunoassay is readily conducted to determine PCB compound levels such as meat, wherein the PCB standards include PCB 118, PCB 138, PCB 153, PCB 180 and combinations thereof. Preferred proportions of the standard PCB compounds include PCB 118:10%; PCB 138:40%; PCB 153:30% and PCB 180:20% relative to total polychlorinated biphenyl compounds in the standardized control.

In a preferred option, the methods of the invention provide a broader process for optimizing an immunoassay according to the invention by preparing a standard composition by:

identifying a type of biological material of interest,

determining species, proportions and concentrations of polychlorinated biphenyls in type of biological material of interest by conducting gas chromatography/mass spectroscopy analysis of representative samples of the biological material of interest;

preparing a standard composition comprising the species of polychlorinated biphenyl compounds identified as present in the biological material of interest. The inventive immunoassay provides determinations of PCB compounds with a much improved correlation to GC/MS, e.g., with a correlation value, or R of at least 0.95; and wherein R² is at least 90, relative to the determination of the same samples by gas chromatography and mass spectroscopy. In addition, the assay can be conducted with relatively small sample volumes, e.g., with a volume ranging from about 5 to about 200 microliters for each ml of nonpolar solvent. Further, when the analyte of interest includes lipids, the volume of the substantially isolated lipid, combined with the volume of the nonpolar solvent, preferably ranges from about 0.5 ml to about 7 ml. The assays are completed more rapidly than GC/MS, e.g., about 80 individual sample determinations are completed in a time ranging from 8 to about 24 hours.

The mAB employed in the inventive immunoassay is prepared, for example, by the method of Friedman et al., supra, which, briefly, consists of:

(i) providing an immune response in a vertebrate host by immunization with an immunogen comprised of a derivative moiety of formula:

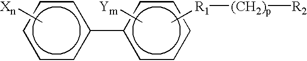

wherein X and Y independently represent a halogen,

n is an integer from 0 to 5,

m is an integer from 0 to 4, wherein n and m cannot both be 0;

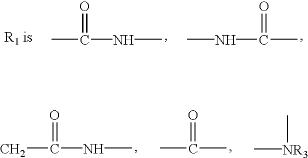

or a single bond,

R₂ is

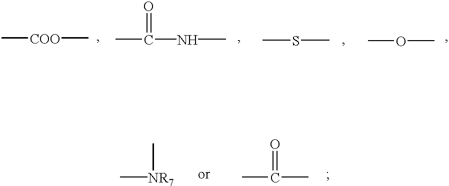

wherein R₃ and R₄ are each independently hydrogen, C₁-C₂ alkyls, linear, branched, or cyclic C₃-C6 alkyls; and p is 0 or an integer from 1 to 4;

linked to an immunogen carrier molecule;

(ii) preparing a hybridoma from the lymphoid cells of said host;

(iii) selecting said hybridoma which produces said monoclonal antibody; and

(iv) obtaining said monoclonal antibody.

The present invention also provides the above-mentioned control compositions, and kits that includes reagents and materials suitable for conducting a Delphia immunoassay, and an mAB that has specific reactivity towards AROCLORs 1260, 1254, 1248, 1242, 1232, 1016 and 1221 that is substantially the same as the mAB produced by clone ATCC No. HB-12421.

### BRIEF DESCRIPTION OF THE FIGURES

FIG. 1 illustrates the calibration curve and precision profile obtained by employing the DELFIA® PCB XL assay using standardized control samples with the following proportions: PCB 118:10%;PCB 138:40%;PCB 153:30% and PCB 180:20%. The Results are expressed as the mean ±SD (n=13).

FIG. 2 illustrates a comparison of PCB concentrations in field samples (pork fat) determined by GC/MS and by the DELFIA® PCB XL assay. Linear regression analysis was performed (Y=27.8+0.895A; Correlation (R)=0.95; R1=90). The values represent single measurements respectively.

### DETAILED DESCRIPTION OF THE INVENTION

The present invention overcomes the previous obstacles to provide rapid, sensitive, accurate and economical immunoassays for the determination of PCB compounds in biological materials.

Broadly, a direct analysis method, GC/MS is employed to determine PCB compounds typically present in a biological material to be tested by immunoassay, which can be considered to be an indirect method of analysis, since standards must be run in order to calibrate the immunoassay results. From the GC/MS data, calibration compositions or standards are prepared, which include the particular PCB compounds, in appropriate proportions and concentrations, respectively. In addition, reliable and reproducible methods of extracting PCB compound contaminants from samples of interest, e.g., biological materials, must be employed.

Definitions

For convenience of description, several terms are defined as follows. The term, “biological material” broadly refers to substances or materials obtained from or part of living or formerly living organisms, whether animal or plant in nature. Thus, and without limitation, the term as employed herein refers to animal products, including those for both human and animal consumption, such as muscle, bone, skin, blood, milk, eggs, bone marrow, organ tissue, and so forth. Other biological materials include milk, and processed milk products, e.g., cheese, yogurt, ice cream, separated milk fractions such as cream, butter and the like. These can be obtained from beef, lamb, pork and other domesticated and wild mammals typically used as food.

The term also refers to plant products such as seeds, e.g., grains, nuts, and the like, fruits, leaves, stems, roots, and substances extracted from any of these materials, such as vegetable oils. Other food sources are animal meats, including beef, lamb, pork and other commonly consumed muscle and organ meats derived from domesticated and wild mammals typically used as food, as well as meat and eggs obtained from fish and poultry. As mentioned above, these materials include food, as well as medicinal and grooming products, for humans and animals.

Of course, the artisan will appreciate that when the biological materials are tested for medical or diagnostic purposes, the sources can include tissue or blood obtained from living animals or even human patients in need of such testing.

While any biological materials are contemplated to be analyzed by the inventive methods, given the lipophilic nature of PCB compounds, biological materials that include a lipid component are preferred.

The term, “lipid” refers broadly to hydrophobic materials or substances that will tend to accumulate PCBs in a living organism, including, without limitation, fats or oils and derivatives thereof, such as fatty acids. The term is also contemplated to encompass lipid derivatives, e.g., phospholipids, or other art-known hydrophobic components of biological materials, in which PCB compounds preferably accumulate.

The phrase, “PCB compounds” broadly refers to any of the specific PCB congeners, and potential degradation products of the same, that may be present in PCB contaminated materials. These include any of the 209 different isomeric forms of PCB found in the commercial Aroclor compositions, as well as others that may have resulted from non-commercial production, or the metabolic actions of living organisms, e.g., biotransformation by soil microorganisms.

Further, the use of singular terms for convenience in description is in no way intended to be so limiting. Thus, for example, reference to a composition comprising “an antibody” includes reference to one or more of such antibodies, e.g., to a preparation with sufficient antibodies for the intended purpose, unless otherwise stated.

The term, “immunoassay” refers to an assay based on the specific reaction, by binding or a selective catalytic reaction, between an antigen and its corresponding antibody. The artisan will appreciate that any suitable type of immunoassay is readily employed in the methods of the invention, although the immunoassay exemplified hereinbelow is preferred. Immunoassays for detecting small molecules called haptens are usually competitive in nature. In this type of assay, a fixed amount of a labelled hapten (tracer) competes with an unlabelled hapten (present in the sample) for a limited number of antibody binding sites. The specific activity of the tracer plays an important role in determining the sensitivity of the assay. Ekins, 1997 (9).

It is also to be understood that this invention is not limited to the particular configurations, process steps, and materials disclosed herein, as such configurations, process steps, and materials may vary somewhat. It is also to be understood that the terminology employed herein is used for the purpose of describing particular embodiments only and is not intended to be limiting, since the scope of the present invention will be limited only by the appended claims and equivalents thereof.

Optimizing PCB Immunoassays

Given the previous failures in the art to achieve rapid, accurate and economical immunoassay methods for determining PCB compounds in biological materials, a different approach has been utilized.

An immunoassay technique does not measure defined physical or chemical parameters, such as moles or grams, directly, but depends on the use of standards with predetermined or arbitrary values assigned to them. Thus, immunoassays are based on an indirect measurement.

The strategy successfully employed herein was to utilize a direct measurement technique to calibrate an indirect measurement technique. Currently, calibration of an indirect assay method requires a source of pure or purified analyte of the highest grade available.

Optionally, an indirect method such as an immunoassay may be calibrated using the signal of the direct method. Any signal-producing reagent may be utilized in the indirect assay that matches the signal generated from the direct method. As such, the concentration of one specific PCB congener could be so adjusted at each selected calibrator concentration to produce a result that mimics the mixture of PCB congeners generally found in the sample of interest, i.e., a type of biological material.

In addition, any chemical species that generates a signal in the indirect method maybe substituted to produce a standard. This provides the benefit of producing a set of non-toxic calibrators. Thus, to illustrate based on the present invention, GC/MS was employed to test 55 samples of pork meat. Once the predominant PCB compounds were identified, a calibration standard that reflected the GC/MS data was provided.

Optionally, instead of using toxic PCB compounds for standardization, nontoxic substitutes that yield an equivalent signal in the immunoassay are substituted. The artisan will appreciate that this strategy has broad potential for measuring trace amounts of many substances, including other environmental toxins with slow rates of degradation and a tendency to accumulate in plants and animals.

Immunoassays and the DELFIA® Method

The commercially available DELFIA® immunoassay was employed in the Examples provided hereinbelow. The DELFIA® immunoassay kit includes the F4011GS mAb and allows a fast and simple semi-quantitative determination of PCBs in the tested sample. Moreover, the method does not require extensive sample cleanup or the large amounts of solvent used by conventional methods thus, ensuring a more cost-effective, simpler and safer operation. The presented method also offers good reliability and repeatability at the PCB concentrations of interest.

The detectable tracer technology employed in the DELFIA® immunoassay is based on time-resolved fluorometry of lanthanide compounds, such as Europium. Lanthanide ions exhibit a unique fluorescence that is characterized by narrowband emission lines, a long decay time, and large Stokes shift. The specific fluorescence of the lanthanide label is measured after a certain time delay following an activation pulse. The delay allows all of the non-specific background to expire (Hemmila 1985). The sensitivity of time-resolved fluorometry reduces the amount of fat required for analysis so that only 2 ml of total solvent are needed per sample analysis.

The PCB immunoassay is sensitive to most PCB congeners. Commercially available Aroclor mixtures are not suitable calibrators for analysis of biological materials, since the congener distribution is biologically altered through preferential transformation and rapid clearance of less chlorinated congeners. Studies have shown that biological matrices tend to accumulate specific PCB congeners that do not resemble commercial samples. Schwartz and Stalling 1987, Id.; Jordan and Feeley 1999, Id. For this reason the test should be calibrated using a congener mix known to be present in food samples of interest.

As described in greater detail in the Examples below, the analysis of 55 meat samples using GC/MS methodology revealed that the predominant congeners were PCB 118, PCB 138, PCB 153, and PCB 180. As described by Frame, (Analytical Standards and Reference Material for all 209 PCB Congeners, Catalog number S-3571, Accustand Inc., New Haven, Conn.) incorporated by reference herein in its entirety, these PCB numbers correspond to the following named PCB compounds.

PCB 118 2,3′,4,4′,5-Pentchlorobiphenyl

PCB 138 2,2′,3,4,4′,5′-Hexachlorobiphenyl

PCB 153 2′,2′,4,4′,5,5′-Hexachlorobiphenyl

PCB 180 2,2′,3,4,4′,5,5′-Heptachlorobiphenyl

From these findings, a PCB-congener mix containing the following congeners was prepared for the subsequent calibration of the immunoassay: PCB 118:10%; PCB 138:40%; PCB 153:30% and PCB 180:20% relative to total PCBs. The standards ranged in concentration from about 1.25 ng/ml to about 15 ng/ml total PCB mix in DMSO (1.25 ppb to 15 ppb).

Moreover, these congeners have been found to be among the most consistently detected and quantitatively dominant congeners in human and animal tissues worldwide. Hanson 1998 (16); Hanson 1999 (15); Jordan and Feeley 1999, Id; Humphry et al. 2000 (18).

PCB-Binding Monoclonal Antibody

The PCB-binding monoclonal antibody exemplified herein is designated as F40-11G6, and is described in detail Friedman, et al. 1998, 1999 (U.S. Pat. Nos. 5,834,222, and 5,858,692), incorporated by reference herein in their entireties. In brief, the F40-11G6 monoclonal antibody has specific reactivity towards AROCLORs 1260, 1254, 1248, 1242, 1232, 1016 and 1221, and is produced by clone ATCC No. HB-12421. It is also contemplated that the F40-11G6 antibody is readily substituted for by another monoclonal antibody that is substantially the same as the monoclonal antibody produced by ATCC No. HB-12421, e.g., a monoclonal antibody with specific binding reactivity to towards AROCLORs 1260, 1254, 1248, 1242, 1232, 1016 and 1221.

Friedman et al. obtained the above-described monoclonal antibody by the general method of:

(i) providing an immune response in a vertebrate host by immunization with an immunogen comprised of a derivative moiety of formula:

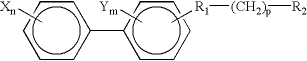

wherein X and Y independently represent a halogen,

n is an integer from 0 to 5,

m is an integer from 0 to 4, wherein n and m cannot both be 0;

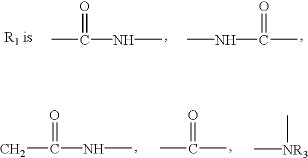

or a single bond,

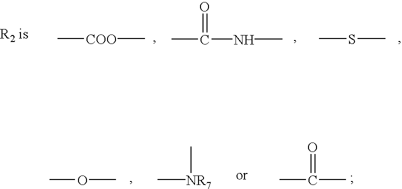

wherein R₃ and R ₄ are each independently hydrogen, C₁-C₂ alkyls, linear, branched, or cyclic C₃-C₆ alkyls; and p is O or an integer from 1 to 4; linked to an immunogen carrier molecule;

(ii) preparing a hybridoma from the lymphoid cells of said host;

(iii) selecting said hybridoma which produces said monoclonal antibody; arid

(iv) obtaining said monoclonal antibody.

### EXAMPLES

Many modifications and variations of this invention can be made without departing from its spirit and scope, as will be apparent to those skilled in the art. The specific embodiments described below are offered by way of example only, and the invention is to be limited only by the terms of the appended claims, along with the full scope of equivalents to which such claims are entitled.

### Example 1

### Gas Chromatography-Mass Spectroscopy

Raw pork was analyzed for PCB content by GC/MS to determine the predominate species of PCB in food and to validate the immunoassay procedure.

A. Sample Material

Fifty-five samples of raw pork meat were analyzed. The samples were of predominantly Belgian origin. Before and after analysis the samples were stored at −18° C. Certified reference material (cod-liver oil and mackerel oil) containing a known amount of PCBs was provided by the Institute of Reference Materials and Measurements of the European Commission (Geel, Belgium). Detailed information about the certification process of this material is given by Griepnik et al. 1988, Report 11520 (14).

B. Sample Preparation

To obtain a homogeneous test portion, an aliquot of the fresh meat was blended with a high-speed blender (Buechi Mixer B-400, Zurich, Switzerland). One to two grams of this homogenized material were placed in a disposable aluminium cup and heated at a maximum temperature of 110° C. to generate liquid fat. AOAC, 1995(3). To remove remaining particles, the liquid fat was filtered through glass woosl. A 30 μl aliquot of the filtered liquid fat was dissolved in 1 ml n-hexane in an Eppendorf brand 1.5 ml microcentrifuge tube followed by the addition of 200 μl of concentrated sulphuric acid. The microcentrifuge tubes were vortexed for 10 seconds and briefly centrifuged to partition the hexane from the aqueous layer. The acid extraction step was repeated with the previously removed clear n-hexane layer. A portion of the n-hexane layer was then dried under a gentle stream of nitrogen, dissolved in an equal volume of dimethyl sulfoxide (DMSO) and used for the PCB XL test kit.

C. Gas Chromatography/Mass Spectroscopy (GC/MS) Analysis

For correlation analysis, each sample was analysed following a method described elsewhere. Von Holst et al. 2000 (24). In brief, the homogenised sample was mixed with sodium sulphate, quartz sand, and a surrogate standard, and transferred into a chromatographic column. The fat portion and the PCBs were extracted from the sample with a mixture of n-hexane and acetone, and the liquid extract was evaporated to a defined volume. The extract was divided into two portions for the subsequent fat determination of the sample and the PCB-analysis. The PCB-analysis included two steps:

(1) removing the fat from the extract with concentrated sulphuric acid, and

(2) quantifying the target PCBs congeners by GC/MS (GC 6890, Hewlett Packard, USA coupled with MSD 5973, Hewlett Packard, USA in the selected ion monitoring mode). Seven indicator PCBs were determined (PCB 28, PCB 52, PCB 101, PCB 118, PCB 138, PCB 153 and PCB 180) and the sum of these congeners were used for the validation of the immunoassay, as described below.

### Example 2

### Immunoassay Methods

A. Materials and Reagents

The DELFIA® PCB XL test kit and all the necessary equipment were provided by Hybrizyme (Raleigh, N.C., USA). Dimethyl sulfoxide was purchased from Sigma (St. Louis, Mo., USA), n-hexane (for residue analysis) from Fluka Chemicals (Buchs, Switzerland) and concentrated sulphuric acid from Scharlau (Barcelona, Spain). All PCB congeners were from Dr. Ehrenstorfer GmbH (Augsburg, Germany).

B. Hybrizyme DELFIA® PCB XL Assay

The assay was performed as described by the manufacturer. Briefly, a 40 μl of sample was incubated with a 160 μl of Tris-buffered (pH 7.8) salt solution with casein and 0.1% sodium azide containing monoclonal anti-PCB antibody. This mixture was incubated in microtiter wells coated with capture antibody for 40 min. The wells were washed 3 times to remove possible matrix interferences (compounds that interfere with the tracer binding to the antibody or that destroy the activity of tracer) and 200 μl of Europium-labeled tracer was added for an additional 15 min. The wells were washed 3 times with wash solution, and 200 μl of Enhancement solution added. After addition of Enhancement solution highly fluorescent chelates were formed. The fluorescence was measured by time-resolved fluorescence using a Victor 2 multilabel counter (Wallac Oy, Turku, Finland). All curve fitting and result calculations were performed automatically using a weighted 4/5-parameter logistic model incorporated in StatLIA immunoassay software (Brendon Scientific, Gross Pointe Farms, Mich., USA).

### Example 3

### Experimental Results For Examples 1 and 2

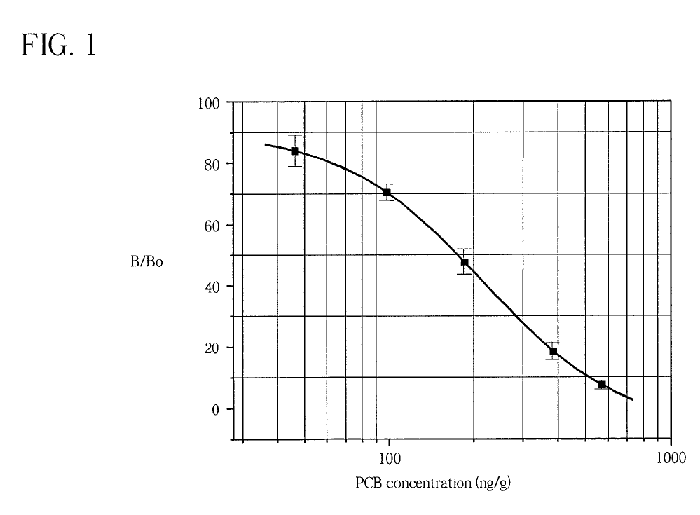

From the data collected from the GC/MS analysis of the 55 meat samples described in Example 1, a calibration curve was established that approximated the distribution and concentration of PCBs in the tested samples. A typical calibration curve using the PCB-congener mix described in this study and precision profile for the DELFIA® PCB assay are shown in FIG. 1.

The precision profile for the immunoassays were calculated from replicate analyses (n=13) on five concentration levels and demonstrated immunoassay coefficients of variation (CVs) of less than 5%. The concentration values applied to the calibration curve reflected the dilution of the sample that occurred during sample processing. The immunoassay itself can detect less than 5 ng/g PCBs in DMSO. The sensitivity of the PCB assay can potentially accommodate a broad range of detection limits by making small changes in sample processing to dilute or concentrate the PCBs.

Analytical Recovery and Accuracy

The PCB-congener mix described above was prepared in hexane and added to fat samples that had previously been determined to be free of PCBs by GC/MS analysis. The samples were spiked at a concentration of 200 ng/g and processed. The average recovery of PCBs for spiked samples was 92% with a standard deviation (SD) of 6% (n=7).

Two different certified reference materials (Griepnik et al. 1988, Ld.) as shown in TABLE 1 were used to examine accuracy of the method and to demonstrate that the PCB congener mix proposed in this study was applicable to the analysis of other food matrices as well The samples were analyzed as described in the experimental section. However, in the last step 200 microliter of n-Hexane was evaporated and resuspended in 1 ml of dimethyl sulfoxide for BCR sample 350 and in 3 ml of dimethyl sulfoxide for BCR sample 349. The dilution of the samples was required in order to adjust the PCB concentration to the linear range of the calibration curve. A comparison of the certified values with the results of the immunoassay showed a good correspondence thereby establishing the correlation of the assay to a common standard. Moreover these results confirm that the above-described congener has a broader field of application, even if the actual distribution of the PCB congeners of the samples under investigation deviates slightly from the PCB distribution of the standard mix.

TABLE 1
Results Of The Assay Using Certified Reference Material.
Mean value and standard deviation (n = 4)

| | BCR* 350 Mackerel oil | BCR* 349 Cod liver oil |
|---|---|---|
| PCB | Certified values ng/g | Certified values ng/g |
| 28 | 22.5 | 68 |
| 52 | 62 | 149 |
| 101 | 164 | 370 |
| 118 | 142 | 454 |
| 138 | 274 | 765 |
| 153 | 317 | 938 |
| 180 | 73 | 280 |
| Sum | 1055 | 3024 |
| Assay | 1002 ± 26 | 3217 ± 200 |
| ng/g | | |

*BCR 349 and 350 are certified reference samples available from the European Commission, consisting of 2 grams of cod liver oil or 2 grams of mackerel oil with endogenous chlorobiphenyls in a sealed argon filled ampoule; the oil is stabilized by the addition of BHT (0.2 mg/g). These are commercially available from the European Commission, Joint Research Centre, Institute for Reference Materials and Measurements Retieseweg, B-2440 Geel, Belgium.

Precision And Detection Limit

The precision profile of the DELFIA® PCB assay system was established using 6 fat samples contaminated with PCBs. Assay precision reflects not only the immunoassay, but also the repeatability of the sample processing protocol. Intra-assay precision was performed using samples ranging in concentration from below 100 ng/g to over 500 ng/g. Each replicate was carried through sample processing and all 10 extracts from each sample set were analyzed by the PCB XL assay in one analytical run. The concentration, SD, and the coefficient of variation (CV) of each set of replicates were determined. As shown by Table 2, below, the immunoassay CVs were less than 16% between 90 and 524 ng/g. As expected, the assay CVs increased as the concentration of PCBs in a sample approached the low-end sensitivity of the assay. The detection limit for the assay was determined by adding the mean result of replicate (n=14) fat samples free of PCBs to 3 times the standard deviation and calculating the corresponding PCB concentration from the calibration curve. The determined value for the detection limit was 48 ng/g.

TABLE 2
Intra-assay Coefficients of Variation (CV) in fat.
The within-day precision was determined by assaying
10 replicates of each sample in one analytical run.

| | Mean | Standard | |
|---|---|---|---|
| | PCB concentration | Deviation | CV |
| Sample | ng/g | ng/g | % |
| 1 | 89.9 | 14.3 | 15.9 |
| 2 | 115.2 | 10.4 | 9.0 |
| 3 | 171.7 | 8.6 | 5.0 |
| 4 | 225.9 | 16.1 | 7.1 |
| 5 | 259.5 | 15.3 | 5.9 |
| 6 | 524.0 | 16.1 | 3.1 |

Inter-assay precision was determined by performing 10 separate PCB analyses, each analysis consisting of a fat sample with low, medium, and high levels of PCBs. The inter-assay precision was calculated from processing and analysing the same set of fat samples on 10 different days, and the results are shown by Table 3, below.

TABLE 3
Inter-assay Coefficient of Variation (CV) in fat.
The between-day precision was determined by assaying
fat samples on 10 different days.

| | Mean | | |
|---|---|---|---|
| | PCB concentration | Standard Deviation | CV |
| Sample | ng/g | ng/g | % |
| 1 | 106.7 | 10.9 | 10.3 |
| 2 | 185.5 | 24.1 | 13.0 |
| 3 | 267.2 | 17.6 | 6.6 |

Estimate of False Negatives at The Action Level by Analysis of PCB-Containing Pork Samples

The concentration of PCBs in 39 meat samples naturally contaminated with PCBs was determined by GC/MS analysis and by the PCB assay. The samples reporting negative or outside the reporting ranges of the immunoassay are shown by Table 4, below.

TABLE 4
Samples having values outside of the quantification
range of the DELFIA ® immunoassay

| | GC/MS | DELFIA ® |
|---|---|---|
| Sample | ng/g | ng/g |
| 1-7 | Non Detect | <48 |
| 8 | 18 | <48 |
| 9 | 23 | <48 |
| 10 | 27 | <48 |
| 11 | 53 | <48 |
| 12 | 109 | <48 |
| 13 | 1943 | >570 |
| 14 | 2194 | >570 |
| 15 | 2972 | >570 |

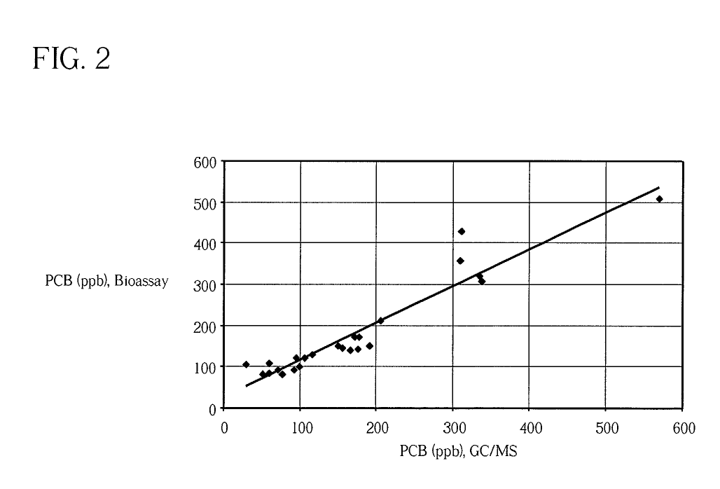

FIG. 2 shows the correlation between GC/MS and the DELFIA® assay. The samples used for the examination of the correlation were pork samples naturally contaminated with PCBs. Linear regression analysis demonstrated an r-squared value of 90.4% (y=0.8945x+27.843, R2=0.9039).

The implementation of the immunoassay system as a rapid and low cost screening method to detect meat samples with 200 ng/g PCBs or greater was examined. Based on the 39 food samples analyzed by GC/MS and the PCB assay an action level was selected for the immunoassay that would provide a large margin of safety in identifying samples contaminated with PCBs. Using the parametric bootstrap procedure. Efron and Tibshirani, 1993 (10), it was estimated that a 0.2% false negative rate was associated with setting the action level at 100 ng/g, to detect samples having 200 ng/g or greater PCBs. The 200 ng/g compliance level is based on a decision of the European Commission on protective measures. European Commission 1999a (12). The action level for the immunoassay would be set to minimize false negative results. All positive samples would then be confirmed with GC/MS analysis.

This analysis assumes a linear relationship between the DELFIA® assay and GC/MS measurements with random, normally distributed errors. The 0.2% estimate of false negatives has a standard error of 0.5% and a 95% upper confidence bound of 1.3%. This value is acceptable since a screening method should have a maximum false negative rate of 5%. European Commission 1999b (13) for the analysis of food according to European legislation.

### Example 4

### Analysis of Animal And Grain Products

The above-exemplified methods were further validated by detecting PCBs in meat samples and cooking grease. In total, 44 samples (including duplicates) of fat and cooking grease were processed and analyzed. Samples included fat from pork, beef, chicken, and cooking grease that had previously been analyzed by Gas Chromatography (GC).

The testing of 44 samples of meat/animal fat employed the following materials.

88 1.5 ml microfuge tubes

44 10×75 mm glass tubes

44 ml of Hexane

8.8 ml of Sulfuric Acid

8.8 ml of DMSO

Disposable pipette tips

Hybrizyme DELFIA® PCB XL assay kit.

A. Processing of Meat/Grease Samples

1. Samples of pork, chicken, beef, and cooking fat was processed into liquid fat.

2. Liquid fat (50 μl) was added to 1000 μl of hexane in disposable 1.5 ml Eppendorf brand microfuge tubes and mixed.

3. Concentrated sulfuric acid (200 μl) was added and each tube was vortexed for 10 seconds.

4. The microfuge tubes were spun at 10,000 rpm for 30 seconds.

5. Approximately 700 μl of hexane was removed and placed in a new Eppendorf microcentrifuge tube.

6. Concentrated sulfuric acid (200 μl) was added and each tube was vortexed for 10 seconds.

7. The microfuge tubes were spun at 10,000 rpm for 30 seconds.

8. Hexane (400 ul) was removed and placed in a 10×75 mm disposable glass tube and dried under a gentle stream of nitrogen (approximately 5 minutes).

9. The dried samples were reconstituted with 400 μl of DMSO.

B. Processing of Grain for Animal Feed

Feed meal (1 gram) was extracted in 5 ml of hexane. The hexane was evaporated under nitrogen to a final volume of 1 ml. The samples were then processed as described above starting with the first addition of sulfuric acid in step 3, supra.

C. Hybrizyme PCB XL ImmunoAssay

The assay was performed according to the instructions provided with each kit, as described in Example 2, supra. Each kit contains reagents for analyzing 40 samples. During incubation with sample and PCB Antibody, any PCB that is present is bound to the antibody. A second antibody, which binds the PCB Antibody, is attached to the microtiter plate wells, and traps the Ab-PCB complex. The first wash step removes matrix interferences that may be in the sample. A Europium-labeled PCB compound (PCB Tracer) is then allowed to bind to any PCB Antibody binding sites that are empty. A wash step separates antibody-bound and free tracer. Following the wash step, the addition of Enhancement Solution forms highly fluorescent chelates with the bound europium ions. The amount of fluorescence measured is inversely proportional to the concentration of PCB in the sample.

D. Results

Analyses of the samples were performed on 2 different days in 4 batches. Duplicates were randomly dispersed throughout the analyses. Correlation with CG was determined using a 200 ppb set threshold for compliance. The Hybrizyme PCB XL had 96% (42 samples) correlation with GC demonstrating 4% (2 samples) false positives.

Analysis of the animal feed (grain) data clearly demonstrated that the PCB XL test could be employed to examine a variety of matrices using The PCB XL standard sample processing procedure.

The data are summarized by Table 5, as follows.

TABLE 5
CORRELATION BETWEEN PCB XL AND GC/MS

| | | PCB XL | PCB XL | | |
|---|---|---|---|---|---|
| No. | GC/MS² | B/Bo³ | Result* | Correlation | Matrix |
| 1 | 3761 | 0.02 | Positive | Correct | Animal Fat |
| 2 | 4208 | 0.02 | Positive | Correct | Animal Fat |
| 3 | 556 | 0.03 | Positive | Correct | Animal Fat |
| 4 | 24 | 0.79 | Negative | Correct | Animal Fat |
| 5 | 3347 | 0.02 | Positive | Correct | Animal Fat |
| 6 | 25 | 0.84 | Negative | Correct | Animal Fat |
| 7 | 53 | 0.65 | Negative | Correct | Animal Fat |
| 8 | 718 | 0.02 | Positive | Correct | Animal Fat |
| 9 | 134 | 0.26 | Positive | False Positive | Animal Fat |
| 10 | 53 | 0.71 | Negative | Correct | Animal Fat |
| 11 | 25 | 0.78 | Negative | Correct | Animal Fat |
| 12 | 228 | 0.21 | Positive | Correct | Animal Fat |
| 13 | 2122 | 0.01 | Positive | Correct | Animal Fat |
| 14 | 228 | 0.19 | Positive | Correct | Animal Fat |
| 15 | 3251 | 0.02 | Positive | Correct | Animal Fat |
| 16 | 3550 | 0.02 | Positive | Correct | Animal Fat |
| 17 | 1655 | 0.02 | Positive | Correct | Animal Fat |
| 18 | 3550 | 0.02 | Positive | Correct | Animal Fat |
| 19 | 718 | 0.02 | Positive | Correct | Animal Fat |
| 20 | 203 | 0.21 | Positive | Correct | Animal Fat |
| 21 | 256 | 0.11 | Positive | Correct | Animal Fat |
| 22 | 3347 | 0.02 | Positive | Correct | Animal Fat |
| 23 | 1153 | 0.02 | Positive | Correct | Animal Fat |
| 24 | 256 | 0.12 | Positive | Correct | Animal Fat |
| 25 | 0 | 0.89 | Negative | Correct | Animal Fat |
| 26 | 3761 | 0.02 | Positive | Correct | Animal Fat |
| 27 | 3272 | 0.02 | Positive | Correct | Animal Fat |
| 28 | 2122 | 0.02 | Positive | Correct | Animal Fat |
| 29 | 100 | 0.69 | Negative | Correct | Animal Fat |
| 30 | 105 | 0.69 | Negative | Correct | Animal Fat |
| 31 | 100 | 0.58 | Negative | Correct | Animal Fat |
| 32 | 718 | 0.04 | Positive | Correct | Animal Fat |
| 33 | 105 | 0.84 | Negative | Correct | Animal Fat |
| 34 | 0 | 0.94 | Negative | Correct | Animal Fat |
| 35 | 2122 | 0.03 | Positive | Correct | Animal Fat |
| 36 | 134 | 0.31 | Positive | False Positive | Animal Fat |
| 37 | 0 | 0.79 | Negative | Correct | Animal Fat |
| 38 | 3251 | 0.02 | Positive | Correct | Animal Fat |
| 39 | 556 | 0.03 | Positive | Correct | Animal Fat |
| 40 | 1153 | 0.02 | Positive | Correct | Animal Fat |
| 41 | 718 | 0.03 | Positive | Correct | Animal Fat |
| 42 | 24 | 0.86 | Negative | Correct | Animal Fat |
| 43 | 718 | 0.03 | Positive | Correct | Animal Fat |
| 44 | 0 | 0.99 | Negative | Correct | Animal Fat |
| 45 | 268 | 0.42 | Positive | Correct | Animal Feed |
| 46 | ND | 0.96 | Negative | Correct | Animal Feed |
| 47 | 364 | 0.34 | Positive | Correct | Animal Feed |
| 48 | ND | 1.03 | Negative | Correct | Animal Feed |
| 49 | ND | 0.98 | Negative | Correct | Animal Feed |
| 50 | 1784 | 0.02 | Positive | Correct | Animal Feed |
| 51 | 92 | 0.71 | Negative | Correct | Animal Feed |

*Positive means equal to or above 200 ppb PCBs and negative means below 200 ppb.
²GC/MS as noted above (and in Table 4) is measured in units of nanogram PCB per gram of the initial sample (ng/g).
³“B/Bo” is the flourescence count of the sample (“B”) normalized against background as measured by “Bo,” which is the raw flouresence count of a DMSO blank. Thus, a B/Bo ratio of 0.4 corresponds to 200 ppb (equivalent to 200 ng/gram) for the fat samples and for the food samples.
⁴Correlation means that when the B/Bo ratio yielded by the immunoassay corresponds to 200 ppb, the GC assay also yielded a value equal to or greater than 200 ppb (ng/g).

The Hybrizyme PCB XL or Delphia® immunoassay allows a technician to test 5-80 samples per day. Table 5 above confirms that the inventive assay is a useful and accurate method for analysis and screening of the PCB content of human and animal foodstuff.

While there have been described what are presently believed to be the preferred embodiments of the invention, those skilled in the art will realize that changes and modifications may be made thereto without departing from the spirit of the invention. It is intended to claim all such changes and modifications that fall within the true scope of the invention. Numerous references are cited in the specification, the disclosures of which are incorporated by reference in their entireties. For those references cited in the specification with an incomplete citation, the following List of References completes the respective citations.

### LIST OF REFERENCES

1. Alcock, R. E., Behnisch, P. A., Jones, K. C., Hagenmaier, H., 1998, Dioxin-like PCBS in the environment—human exposure and the significance of sources. Chemosphere, 37,1457-1472.

2. Allen, R. L., Manning, W. B., McKenzie, K. D., Withers, T. A., Mapes, J. P., Friedman, S.B ., 1992, Development of a Monoclonal Antibody Immunoassay for the Detection of Gasoline and Diesel Fuel in the Environment. Contaminated Soils, Diesel Fuel Contamination, edited by Kostecki, P. T. and Calabrese, E. J. (New York, USA: Lewis Publishers) pp. 37-46.

3. AOAC, 1995, Official Method No. 984.21, Organochlorine Pesticide Residues in Animal Fats

4. Battershill, J. M., 1994, Review of the safety assessment of polychlorinated biphenyls (PCBs) with particular reference to reproductive toxicity. Human Exp. Toxicol. 13, 581-597.

5. Bernard, A., Hermans, C., Broeckaert, F., De Porter, G. De Cock, A., and Housins, G., 1999, Food contamination by PCBs and dioxins. Nature, 401, 231-232.

6. CEN 1997, EN 1528-1-4, Fatty Food. Determination of pesticides and polychlorinated biphenyls (PCBs). General; Extraction of fat, pesticides and PCBs, and determination of fat content; Clean-up methods; Determination, confirmatory tests, miscellaneous

7. Cheng, Y. J., and Hsu, C. C., 1994, Effects of prenatal exposure to PCBs on the neurological function of children: A neuropsychological and neurophysiological study. Dev. Med. Child Neurol. 36, 312-320.

8. Deutsche Forschungsgemeinschaft, Mitteilung XIII der Senatskommisssion zur Pruefing von Rueckstaenden in Lebensmittel, 1988, Polychlorierte Biphenyle, Bestandsaufnahme ueber Analytik, Vorkommen, Kinetik und Toxikologie. (Weinheim, Germany: VCH Verlagsgesellschaft mbH

9. Ekins, R. P., 1997, Immunoassay Design and Optimisation. Principal and Practice of Immunoassay, 2^{nd} Edition, edited by Price, C. P and Newman, D. J. (New York, USA: Stockton Press).

10. Efron, B, and Tibshirani, R. J., 1993, An introduction to the Bootstrap (New York, USA: Chapman and Hall).

11. Erickson, M. D., 1997, Analytical Chemistry of PCBs. (Boca Raton, USA: CRC Press), pp. 18-20.

12. European Commission, 1999a, Commission decision 1999/788/EC on protective measures with regard to contamination by dioxins of certain products of porcine and poultry origin intended for human or animal consumption. Official Journal of the European Communities, L 310/62, 62-70.

13. European Commission, 1999b, Commission Decision laying down analytical methods to be used for detecting certain substances and residues thereof in live animals and animal products according to Council Directive 96/23/EC (Revision of Commission Decision 93/256/EC) Final version.

14. Griepnik B., Wells D. E., Frias Ferreira M., 1988. The certification of the contents (mass fraction) of chlorobiphenyls (IUPAC Nos 28, 52, 101, 118, 138, 153, 180) in two fish oils. Cod-liver oil CRM No 349, Mackerel oil CRM No 350. (EU Report 11520 en) Luxembourg: Office for Official Publications of the European Communities)

15. Hanson, L. G., 1999, The Ortho Side of PCBs: Occurrence and Disposition (Boston, Mass., USA: Kluwer Academic Publishers).

16. Hanson L. G., 1998, Stepping backward to improve assessment of PCB congener toxicities. Environ. Health Perspect. 106 (suppl 1), 171-189.

17. Hemmilä, I., 1985, Fluoroimmunoassays and immunofluorometric assays. Clin. Chem. 31, 1677-1681.

18. Humphry, H. E. B., Gardiner, J.C., Pandya, J. R., Sweeney, A. M., Gasior, D. M., McCaffrey, R. J., Schantz, S. L., 2000, PCB Congener Profile in the Serum of Humans Consuming Great Lakes Fish. Environ. Health Perspect. 108, 167-172.

19. Jordan, S. A, and Feeley, M. M., 1999, PCB Congener Patterns in Rats Consuming Diets Containing Great Lakes Salmon: Analysis of Fish, Diets, and Adipose Tissue. Environ. Res. Section A, 80, S207- S212.

20. Kimbrough, R., 1993, Polychlorinated biphenyls (PCB) and human health: An update. Crit. Rev. Toxicol. 25, 133-163.

21. Mapes, J. P., McKenzie, K. D., Stewart, T. N., McClelland, L. R., Studabaker, W. B., Manning, W. B., Friedman, S. B., 1993, An on-site immunoassay for detecting PCB in soil. Bull. Environ. Contam. Toxicol., 50, 219-225.

22. Seegal, R. F. and Shain, W., 1992, Neurotoxicity of Polychlorinated Biphenyls, The Role of Ortho-substituted Congeners in Altering Neurochemical Function. Toxins in Food—The Vulnerable Brain and Environmental Risks, Volume 2, edited by R. L. Isaacson and K. F. Jensen (New York, USA: Plenum Press), pp.173-191.

23. Schwartz, T. R., and Stalling, D. L., 1987, Are polychlorinated biphenyl residues adequately described by Aroclor mixture equivalents? Isomer-specific principal component analysis of such residues in fish and turtles. Environ. Sci. Technol. 21, 72-76.

24. von Holst C, Müller A, Anklam E, 2000. Determination of Polychlorinated Biphenyls (PCBs) in food and feedingstuffs samples by GC/MS. European Commission, EU-report EUR 19571 EN

25. Withers, T., Almond, R., Friedman, S., Stewart, T., Allen, R., 1995, Benzene RISc®: An Immunoassay for Detecting 500 Parts per Billion Benzene in Water. J. of Clinical Ligand Assay, 18, 156-160.

We claim:

1. An immunoassay for determining the presence of polychlorinated biphenyl compounds, in a sample of interest, the process comprising the steps of:
(a) extracting polychlorinated biphenyl compounds present in the sample of interest into a nonpolar solvent to produce an analyte extract,
(b) contacting the analyte extract with a monoclonal antibody with specific reactivity towards polychlorinated biphenyl compounds under conditions suitable for the monoclonal antibody to bind to polychlorinated biphenyl compounds present in the sample,
(c) measuring monoclonal antibody that is selectively bound to polychlorinated biphenyl compounds and congeners thereof, to provide a binding level,
(d) relating the binding level of the monoclonal antibody of step (c) with binding levels of the same monoclonal antibody with a control composition, wherein the control composition comprises a plurality of polychlorinated biphenyl compounds of known concentration, and then determining concentrations of polychlorinated biphenyl compounds in the sample of interest; provided that the nonpolar solvent solubilizes polychlorinated biphenyl compounds.

2. The immunoassay of claim 1 wherein the sample of interest comprises a lipid phase.

3. The immunoassay of claim 2 wherein the polychlorinated biphenyl compounds are extracted into the nonpolar solvent from the lipid phase of the sample of interest by a process comprising
(i) treating the sample of interest by a method selected from the group consisting of, homogenizing the sample, heating the sample, and a combination thereof, in the presence of the nonpolar solvent;
(ii) separating the nonpolar solvent from the treated sample of interest;
(iii) removing dissolved or suspended lipid from the nonpolar solvent to provide an analyte extract comprising the nonpolar solvent and any extracted polychlorinated biphenyl compounds.

4. The immunoassay of claim 2 wherein the polychlorinated biphenyl compounds are extracted into the nonpolar solvent from the lipid phase of the sample of interest by a process comprising
(i) separating the lipid phase from the sample of interest by a method selected from the group consisting of, homogenizing the sample, heating the sample, and a combination thereof and collecting the separated lipid,
(i) dissolving the separated lipid in a nonpolar solvent,
(ii) removing dissolved or suspended lipid from the nonpolar solvent to provide an analyte extract comprising the nonpolar solvent and any extracted polychlorinated biphenyl compounds.

5. The immunoassay of claim 3 wherein step (iii) comprises mixing the nonpolar solvent with a strongly acidified polar solvent, under conditions effective to render the lipid phase of the nonpolar solvent soluble or miscible in the polar solvent, and removing the lipid phase in the polar solvent; provided that the nonpolar solvent is substantially immiscible with the strongly acidic polar solvent.

6. The immunoassay of claim 4 wherein step (ii) comprises mixing the nonpolar solvent with a strongly acidified polar solvent, under conditions effective to render the lipid phase soluble or miscible in the polar solvent, and removing the lipid phase in the polar solvent; provided that the nonpolar solvent is substantially immiscible with the strongly acidic polar solvent.

7. The immunoassay of claim 6 wherein the strongly acidic polar solvent is aqueous concentrated sulfuric acid.

8. The immunoassay of claim 1 wherein the nonpolar solvent is selected from the group consisting of a straight or branched alkyl, substituted or nonsubstuted, an aryl, substituted or nonsubstuted, and combinations thereof, wherein the alkyl moiety ranges in size from about C₃ through about C₂₀.

9. The immunoassay of claim 1 wherein the nonpolar solvent is selected from the group consisting of hexane, isooctane, heptane, ethyl acetate, diisopropyl ether, diethyl ether, dichloromethane, dichloroethane, cyclopentane, cyclohexane, chloroform, carbon tetrachloride
n-butanol, butyl acetate, benzene, pentane, methyl t-butyl ether, trichloroethylene, toluene,
ether and combinations thereof.

10. The immunoassay of claim 1 wherein the binding level of step (c) is determined by conducting a dissociation-enhancement immunoassay by:
(i) incubating the analyte extract with the anti-PCB monoclonal antibody in chambers coated with capture antibody,
(ii) washing the microtiter wells, and
(iii) adding a signal producing reagent, and determining the signal.

11. The immunoassay of claim 10 wherein the dissociation-enhancement immunoassay is a lanthanide fluoro-immunoassay.

12. The immunoassay of claim 11 wherein the signal producing reagent is Europium.

13. The immunoassay of claim 1 wherein the sample of interest is a foodstuff derived from a vegetable or animal source, and the standardized control samples comprise polychlorinated biphenyl compounds of a type and approximate concentration as determined by gas chromatography and mass spectroscopy to be present in the type of foodstuff to be tested.

14. The immunoassay of claim 13 wherein the sample of interest is meat and the standardized control samples comprise polychlorinated biphenyl compounds selected from the group consisting of PCB 118, PCB 138, PCB 153, PCB 180 and combinations thereof.

15. The immunoassay of claim 14 wherein the standardized control comprises the following proportions: PCB 118:10%; PCB 138:40%; PCB 153:30% and PCB 180:20% relative to total polychlorinated biphenyl compounds in the standardized control.

16. A method of preparing a composition suitable to provide a standard for immunoassay determination of polychlorinated biphenyls in a biological material comprising:
identifying a type of biological material of interest,
determining species, proportions and concentrations of polychlorinated biphenyls in type of biological material of interest by conducting gas chromatography/mass spectroscopy analysis of representative samples of the biological material of interest;
preparing a standard composition comprising the species of polychlorinated biphenyl compounds identified as present in the biological material of interest.

17. The immunoassay of claim 1 that determines the concentrations of polychlorinated biphenyl compounds with a correlation value, or R of at least 0.95; and wherein R² is at least 90, relative to the determination of the same samples by gas chromatography and mass spectroscopy.

18. The immunoassay of claim 4 wherein each sample of separated lipid has a volume ranging from about 5 to about 200 microliters for each ml of nonpolar solvent.

19. The immunoassay of claim 4 wherein the volume of the substantially isolated fat combined with the volume of the nonpolar solvent ranges from about 0.5 ml to about 7 ml.

20. The immunoassay of claim 1 wherein 80 individual sample determinations are completed in a time ranging from 8 to about 24 hours.

21. The immunoassay of claim 1 wherein the analyte extract is in contact with the monoclonal antibody for a period of about 5 to about 60 seconds.

22. The immunoassay of claim 1 wherein the monoclonal antibody has specific reactivity towards AROCLORs 1260, 1254, 1248, 1242, 1232, 1016 and 1221 that is substantially the same as the monoclonal antibody produced by clone ATCC No. HB-12421.

23. The immunoassay of claim 22, wherein the antibody is obtained by
(i) providing an immune response in a vertebrate host by immunization with an immunogen comprised of a derivative moiety of formula: 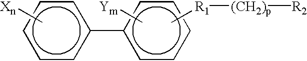
wherein X and Y independently represent a halogen,
n is an integer from 0 to 5,
m is an integer from 0 to 4, wherein n and m cannot both be 0; 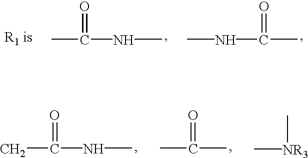
or a single bond,
R₂ is 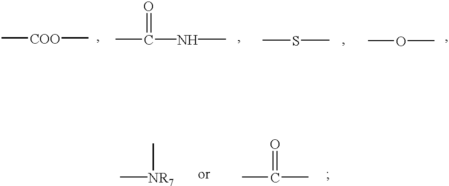
wherein R₃ and R ₄ are each independently hydrogen, C₁-C₂ alkyls, linear, branched, or cyclic C₃-C₆ alkyls; and p is 0 or an integer from 1 to 4;
linked to an immunogen carrier molecule;
(ii) preparing a hybridoma from the lymphoid cells of said host;
(iii) selecting said hybridoma which produces said monoclonal antibody; and
(iv) obtaining said monoclonal antibody.

24. A composition suitable for use as a control reagent for the determination of polychlorinated biphenyl compounds in biological materials that comprises the following polychlorinated biphenyl compounds: PCB 118; PCB 138; PCB 153 and PCB 180.

25. The composition of claim 24 of the following percentages: PCB 118:10%; PCB 138:40%; PCB 153:30% and PCB 180:20%, at a concentration ranging from about 1 to about 100 ng/ml.

26. A kit comprising the composition of claim 24, reagents for conducting a Delphia immunoassay, and a monoclonal antibody that has specific reactivity towards AROCLORs 1260, 1254, 1248, 1242, 1232, 1016 and 1221 that is substantially the same as the monoclonal antibody produced by clone ATCC No. HB-12421.
